SEQUENCE LISTING (4 sequences)

SEQ ID NO: 1 - protein, 585 aa, Bacillus licheniformis
MRRLAFLVVAFCLAVGCFFSPVSKAEGVMSGGGGDKVAVGKDGMVATAHPLASKIGAEVLKKGGNAIDAAIAIQYALNVTEPMMSGIGGGGFMMVYDGETKETSIINSRERAPEGAKPDMFLDGDGKVIPFSERSRHGNAVGVPGTLKGLEAAHKKWGTKKMEDLISPSIKLAEEGFPIDSVLADAIKDHQDKLSKTAAKDIFLPDGEPLKEGDILVQKDLAKTFKLIRKEGSKAFYDGEIGRAIADVVQDFGGSMTPDDLSRYEVTTDKPIWGEYHGYDIASMPPPSSGGVFMLQMLKLIDDFHLSQYDPKSFEKYHLLAETMHLSYADRAAYAGDPEFVDVPLRGLLDPDYIKERQKLISLDSMNRDVKEGDPWKYEEGEPNYEIVPQPEDKTIGETTHFTVTDQWGNVVSYTTTIEQLFGTGILVPGYGLFLNNELTDFDAVPGGANEVQPNKRPLSSMTPTIVFKDEKPVLTVGSPGGTTIIASVFQTILNYFEYGMSLQDAIEEPRIYTNSLTSYRYESGMPEDVRRKLNDFGHKFGANPVDIGNVQSIFIDRENKTFMGVADSSRNGTAVGVNIKTSAK

SEQ ID NO: 2 - protein, 558 aa, Bacillus horikoshii
QKPVKGSNEVAVGKDGMVSTSHPLASEIGADILRKGGNAMDAAIAVQFALNVVEPMMSGIGGGGFMMVYDAETDETTIVNSRERAPAGATPDMFLNPDGSLIPFQERVRHGNSVGVPGTLKGLEAAHEKWGTRPFQQLITPAFQLAQNGFSVDRQLALQIENNKEKLAGTAAKEVFLPKGEPIKEGDWLVQKDLAKTFKLIRSHGSEVFYDGEIGEALAATVQDFGGSMTIEDLQNYGVTEDEPVWGEYKGYDIASMPPPSSGGLFLLQMLKTLDSFDISQYDRRSKEVYHLLAEAMHLSYADRGAYAGDPEFVEVPMIGLLHPDYIAERSALIDINSVNTNPQPGDPWQYEDVDPNYNVIKQNDEKDIGETTHFTVADRWGNLVSYTTTIEQVFGSGIMVPGYGFMLNNELTDFDARPGGANEVQPNKRPLSSMTPTIVFEDGKPIMSVGSPGGPTIITSVLQVVLNVMDYEMGLEEAIAEPRIYTNTINSYRYEDGISAEVLSELNAMGHRFPSNSELIGNVQSILIDYEKDEYVGVADARRDGASVGYTRPGKRK

SEQ ID NO: 3 - DNA, 61 bp, Artificial sequence
  Primer
gactgaattcgagctctataaaaatgaggagggaaccgaatgagacggttagctttcttag

SEQ ID NO: 4 - DNA, 41 bp, Artificial sequence
  Primer
gactggatccacgcgttactatttagccgatgtcttaatgt

(19) United States
(12) Patent Application Publication
(10) Pub. No.: US 2020/0196617 A1
(43) Pub. Date: Jun. 25, 2020

(54) Dough Relaxation Using Gamma Glutamyl Transpeptidase

(71) Applicant: Novozymes A/S, Bagsvaerd (DK)

(72) Inventors: Lisbeth Kalum, Vaerloese (DK); Sara Maria Landvik, Vedbaek (DK); Irina Victorovna Matveeva, Moscow (RU); Steen Troels Joergensen, Alleroed (DK); Kenneth Jensen, Oelsted (DK)

(73) Assignee: Novozymes A/S, Bagsvaerd (DK)

(21) Appl. No.: 16/621,472

(22) PCT Filed: Jun. 20, 2018
(86) PCT No.: PCT/EP2018/066427

§ 371 Date: Dec. 11, 2019

(30) Foreign Application Priority Data
Jun. 22, 2017 (EP) 17177340.1

(51) Int. Cl. A21D 8/04; A21D 13/43; A21D 10/00; C12N 9/10

(52) U.S. Cl. CPC A21D 8/042; A21D 13/43; C12Y 203/02002; C12N 9/104; A21D 10/005

(57) ABSTRACT

A method for improving the extensibility of a dough comprising a) adding a gamma glutamyl transpeptidase to flour or to a dough comprising a flour; and b) making the dough. The gamma glutamyl transpeptidase may be obtainable from Bacillus licheniformis and from Bacillus horikoshii.

### REFERENCE TO A SEQUENCE LISTING

This application contains a Sequence Listing in computer readable form, which is incorporated herein by reference.

### FIELD OF THE INVENTION

The present invention relates to a process for improving the extensibility of dough, e.g., flattened dough, when producing, e.g., bread, flat bread, crackers, pizzas, pasta, noodles, laminated baking products, biscuits, baguettes, and hamburgers.

### BACKGROUND OF THE INVENTION

Today, in the industrial dough-making processes, it is known to add dough-improving additives to the dough in order to improve parameters such as texture, volume, extensibility, and machine ability of the dough.

Reducing agents such as gluthathione, cysteine, malt, protease, sorbic acid, and non-leavening yeast are known dough-improving additives used to improve the extensibility of the dough.

There is still a need for finding improved extensibility solutions in the dough production, without, or only little effect, on other dough parameters, when making products such as bread, flat bread, crackers, pizzas, pasta, noodles, laminated baking products, biscuits, baguettes, and hamburgers.

### SUMMARY OF THE INVENTION

Surprisingly, the inventors have found that gamma glutamyl transpeptidase (E.C. 2.3.2.2) increases the extensibility of a dough without, or very little, effect on other dough parameters, so we claim:

A method for improving the extensibility of a dough comprising

a) adding a gamma glutamyl transpeptidase to flour or to a dough comprising a flour; and

b) making the dough.

In one embodiment, a flattened dough is produced from the dough.

In one embodiment, the dough is made into an edible product selected from the group consisting of bread, flat bread, crackers, pizzas, pasta, noodles, laminated baking products, biscuits, baguettes, and hamburgers.

In one embodiment, the flour is selected from the group consisting of wheat flour, corn flour, rye flour, barley flour, oat flour, rice flour, sorghum flour, and a combination thereof.

In one embodiment, the gamma glutamyl transpeptidase is a bacterial gamma glutamyl transpeptidase, in particular a Bacillus gamma glutamyl transpeptidase.

In one embodiment, the gamma glutamyl transpeptidase has at least 60% identity with SEQ ID NO:1.

In one embodiment, the gamma glutamyl transpeptidase is added in an amount of 0.01-100 mg of enzyme protein per kg of flour.

In one embodiment, additionally glutathione is added to the dough.

In one embodiment, the dough has an extensibility which is better than the extensibility of a dough which is prepared under the same conditions, but without treatment with a gamma glutamyl transpeptidase.

In one embodiment, the dough further comprises one or more enzymes selected from the group consisting of amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucoamylase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase.

In one embodiment, the flat bread is selected from the group consisting of tortillas, pita, Arabic bread, and Indian flat bread, including wheat and gluten free flat bread.

In one embodiment, a premix comprising gamma glutamyl transpeptidase (E.C. 2.3.2.2) and flour is claimed.

In one embodiment, the premix further comprises one or more enzymes selected from the group consisting of amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucanase, galactanase, alpha-galactosidase, beta-galactosidase, glucoamylase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase.

In one embodiment, use of a gamma glutamyl transpeptidase (E.C. 2.3.2.2) for increasing the extensibility of a dough is claimed.

In one embodiment, we claim a composition comprising a gamma glutamyl transpeptidase (E.C. 2.3.2.2), wherein the gamma glutamyl transpeptidase has at least 60% identity with SEQ ID NO:1.

### DETAILED DESCRIPTION OF THE INVENTION

### Definitions

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

Variant: The term “variant” means a polypeptide having gamma glutamyl transpeptidase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position.

Improved property: When the gamma glutamyl transpeptidase, according to the invention, is incorporated into a flour and/or a dough in effective amounts, one or more properties are improved compared to a flour and/or a dough in which the enzyme is not added.

The improved property may be determined by comparison of a dough and/or a baked product prepared with and without addition of the enzyme of the present invention in accordance with the methods described below.

Organoleptic qualities may be evaluated using procedures well established in the baking industry, and may include, for example, the use of a panel of trained taste-testers.

Improved extensibility: The term “improved extensibility of the dough” is defined herein as the property of dough that can be subjected to increased stretching without rupture.

The increased stretching is a very important parameter as it means that it is possible to, e.g., obtain very thin doughs.

Increased strength: The term “increased strength of the dough” is defined herein as the property of dough that has generally more elastic properties and/or requires more work input to mould and shape.

Increased elasticity: The term “increased elasticity of the dough” is defined herein as the property of dough which has a higher tendency to regain its original shape after being subjected to a certain physical strain.

Increased stability of the dough: The term “increased stability of the dough” is defined herein as the property of dough that is less susceptible to mechanical abuse thus better maintaining its shape and volume and is evaluated by the ratio of height: width of a cross section of a loaf after normal and/or extended proof.

Reduced stickiness of the dough: The term “reduced stickiness of the dough” is defined herein as the property of a dough that has less tendency to adhere to surfaces, e.g., in the dough production machinery, and is either evaluated empirically by the skilled test baker or measured by use of a texture analyzer (e.g., TAXT2) as known in the art.

Improved machine ability: The term “improved machine ability of the dough” is defined herein as the property of a dough that is generally less sticky and/or more firm and/or more elastic.

Increased volume of the dough/the baked product: The term “increased volume of the dough/baked product” is measured as the volume of a dough or the volume of a given loaf of bread. The volume may, e.g., be determined by the rape seed displacement method, or by a skilled baker, or by using a Volscan profiler 600 as described in Example 2.

Improved crumb structure of the baked product: The term “improved crumb structure of the baked product” is defined herein as the property of a baked product regarding crumb uniformity, cell wall thickness, and the size of the individual gas cells pores on the slice of bread.

The crumb structure of the baked product is usually evaluated visually by the baker or by digital image analysis as known in the art (e.g., C-cell, Calibre Control International Ltd, Appleton, Warrington, UK).

Improved softness of the baked product: The term “improved softness of the baked product” is the opposite of “firmness” and is defined herein as the property of a baked product that is more easily compressed and is evaluated either empirically by the skilled test baker or measured by use of a texture analyzer (e.g., TAXT2 or TA-XT Plus from Stable Micro Systems Ltd, surrey, UK) as known in the art.

### Dough Compositions for Making Dough

As used herein “dough” means any dough used to prepare a baked or cooked product.

According to the present invention, the dough used to prepare a baked or cooked product may be made from any suitable dough ingredients comprising flour.

As used herein, a “flattened dough” means a dough, which typically has a thickness of one millimeter to a few centimeters.

According to the invention, the flattened dough may be used for making, e.g., flat bread, crackers, pizzas, pasta, noodles, laminated doughs, and biscuits.

A flat bread may be made from a simple mixture of flour, water, and salt and then thoroughly rolled into flattened dough. Flat bread has a very quick baking time (often <2 minutes).

The flat bread may be unleavened, i.e., made without a yeast, or leavened, e.g., made with a yeast.

The flat bread may include further optional ingredients, such as olive oil, sesame oil, shortenings, and spices.

Examples of flat bread include tortilla, pita, Arabic bread, and Indian flat bread, including wheat and gluten free flat bread.

Further non-limiting examples of flat bread include lavash, baladi, barbari, sangak, tandoor, taftoon, shami, halabi, mafrood, burr, bairuti, pocket bread, naan, phulka, chapatti, paratha, Arabic pita, Lebanese, mafrood, hapati, sangak, roti, taboon, shrak, mashrouh, nasir, tannoor, lavash, and taftan.

The dough used to prepare a flat bread product may be made from any suitable flour source, e.g., flour sourced from grains, such as, wheat flour, corn flour, rye flour, barley flour, oat flour, rice flour, or sorghum flour, potato flour, soy flour, flour from pulses, and combinations thereof.

Any flat bread process may be used to prepare the flat bread. The process of preparing flat bread generally involves the sequential steps of dough making (with an optional proofing step), sheeting or dividing, shaping and/or rolling, and proofing the dough, which steps are well known in the art.

The flattened dough according to the invention may also be used to make pizzas. Pizza is a yeasted flatbread typically topped with, e.g., tomato sauce and cheese and baked in an oven.

The flattened dough according to the invention may also be used to make crackers. A cracker is a baked food product made from a flattened dough. Flavorings or seasonings, such as salt, herbs, seeds, and/or cheese, may be added to the dough or sprinkled on top before baking as known in the art. Crackers come in many shapes and sizes—round, square, triangular, etc. Crackers are a kind of ancient flat bread.

The flattened dough according to the invention may also be used to make noodles and pasta.

Noodles are made from unleavened dough which is stretched, extruded, or rolled flat and cut into one of a variety of shapes. Noodles are usually cooked in boiling water, sometimes with cooking oil and/or salt added. They may be pan-fried or deep-fried.

Pasta is typically a noodle made from an unleavened dough of a durum wheat flour mixed with water and/or eggs and formed into sheets or various shapes, then cooked by boiling. Pasta can also be made with flour from other cereals or grains.

The flattened dough according to the invention may also be used to make laminated baking products.

A laminated dough is a culinary preparation consisting of many thin layers of dough separated by butter, produced by repeated folding and rolling. Such doughs may contain many layers, i.e., more than 10 layers. During baking, the water in the butter vaporizes and expands, causing the dough to puff up and separate, while the lipids in the butter essentially fry the dough, resulting in a light, flaky product. Examples of laminated doughs include Croissant pastry, and other pastries such as Danish pastry, Flaky pastry, and Puff pastry.

The flattened dough according to the invention may also be used to make biscuits.

The dough according to the invention may be used to produce any baked or cooked product, in particular bread, flat bread, crackers, pizzas, pasta, noodles, laminated baking products, biscuits, baguettes, and hamburgers.

The dough according to the present invention may also comprise other conventional dough relaxation ingredients such as glutathion, protease, malt, sorbic acid, L-cysteine, and/or yeast extract.

There may be a synergistic effect between gamma glutamyl transpeptidase and glutathion.

There may be a synergistic effect between gamma glutamyl transpeptidase and protease.

There may be a synergistic effect between gamma glutamyl transpeptidase and malt.

There may be a synergistic effect between gamma glutamyl transpeptidase and sorbic acid.

There may be a synergistic effect between gamma glutamyl transpeptidase and L-cysteine.

There may be a synergistic effect between gamma glutamyl transpeptidase and yeast extract.

The dough according to the invention may also comprise one or more emulsifiers. Emulsifiers also serve to improve dough extensibility. Examples of suitable emulsifiers are mono- or diglycerides, polyoxyethylene stearates, diacetyl tartaric acid esters of monoglycerides, sugar esters of fatty acids, propylene glycol esters of fatty acids, polyglycerol esters of fatty acids, lactic acid esters of monoglycerides, acetic acid esters of monoglycerides, lecithin or phospholipids, or ethoxylated monoglycerides. Particular emulsifiers include monoglycerides, diacetyl tartaric acid esters of monoglyceride (DATEM) and sodium stearoyl lactylate (SSL).

Other conventional ingredients that may be added to the dough include proteins, such as milk powder, gluten, and soy; eggs (either whole eggs, egg yolks or egg whites); an oxidant such as ascorbic acid, potassium bromate, potassium iodate, azodicarbonamide (ADA), ammonium persulfate or potassium persulfate; a sugar such as sucrose, dextrose, etc.; a salt such as sodium chloride, calcium acetate, sodium sulfate or calcium sulfate, diluents such silica dioxide, starch of different origins. Still other convention ingredients include hydrocolloids such as CMC, guar gum, xanthan gum, locust bean gum, etc. Modified starches may be also used.

The dough according to the present invention may be a fiber dough, e.g., the dough may contain grains, e.g., whole wheat, and/or are enriched with extra fibres in the form of, e.g., cereal bran, e.g., wheat bran. Wheat bran is produced as a side product of milling wheat into white flour.

Normally, fibres are divided into fine fibres, medium fibres, and coarse fibres as known in the art. Fine fibres are particularly useful in the present invention.

In addition to preparing fresh flattened dough or fresh flattened dough products, the present invention is also directed to a method for preparing a frozen flattened dough or a frozen flattened dough product.

The present invention is particularly useful for preparing flattened dough and products obtained from flattened dough in industrialized processes, where the products are prepared mechanically using automated or semi-automated equipment.

### Enzymes

### Gamma Glutamyl Transpeptidase

Gamma glutamyl transpeptidase (E.C. 2.3.2.2) plays a major role in glutathione metabolism where the enzyme catalyzes the transfer of the gamma glutamyl group from gamma glutamyl compounds to amino acids, peptide acceptors, or water (Tate and Meister, 1981, Mol. Cell. Biochem. 39: 357-368). For example, gamma glutamyl transpeptidase catalyzes the hydrolysis of glutathione to produce glutamic acid, and the transfer of the gamma-glutamyl group of glutathione to an amino acid.

Gamma glutamyl transpeptidases have been reported from, e.g., Bacillus subtilis (JP 4281787), Bacillus natto (JP 2065777), and Bacillus agaradhaerens (WO 02/077009).

According to the present invention, a preferred gamma glutamyl transpeptidase is a bacterial gamma glutamyl transpeptidase; in particular a Bacillus gamma glutamyl transpeptidase; in particular a Bacillus licheniformis or a Bacillus horikoshii gamma glutamyl transpeptidase.

Preferably, the gamma glutamyl transpeptidase is an enzyme having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the polypeptide of SEQ ID NO: 1.

In one embodiment, the gamma glutamyl transpeptidase is an enzyme having the amino acid sequence shown in SEQ ID NO:1 herein:

MRRLAFLVVA FCLAVGCFFS PVSKAEGVMS GGGGDKVAVG
KDGMVATAHP LASKIGAEVL KKGGNAIDAA IAIQYALNVT
EPMMSGIGGG GFMMVYDGET KETSIINSRE RAPEGAKPDM
FLDGDGKVIP FSERSRHGNA VGVPGTLKGL EAAHKKWGTK
KMEDLISPSI KLAEEGFPID SVLADAIKDH QDKLSKTAAK
DIFLPDGEPL KEGDILVQKD LAKTFKLIRK EGSKAFYDGE
IGRAIADVVQ DFGGSMTPDD LSRYEVTTDK PIWGEYHGYD
IASMPPPSSG GVFMLQMLKL IDDFHLSQYD PKSFEKYHLL
AETMHLSYAD RAAYAGDPEF VDVPLRGLLD PDYIKERQKL
ISLDSMNRDV KEGDPWKYEE GEPNYEIVPQ PEDKTIGETT
HFTVTDQWGN VVSYTTTIEQ LFGTGILVPG YGLFLNNELT
DFDAVPGGAN EVQPNKRPLS SMTPTIVFKD EKPVLTVGSP
GGTTIIASVF QTILNYFEYG MSLQDAIEEP RIYTNSLTSY
RYESGMPEDV RRKLNDFGHK FGANPVDIGN VQSIFIDREN
KTFMGVADSS RNGTAVGVNI KTSAK

In another embodiment, the gamma glutamyl transpeptidase is an enzyme having the amino acid sequence shown in SEQ ID NO:2 herein:

QKPVKGSNEVAVGKDGMVSTSHPLASEIGADILRKGGNAMDAAIAVQFAL
NVVEPMMSGIGGGGFMMVYDAETDETTIVNSRERAPAGATPDMFLNPDGS
LIPFQERVRHGNSVGVPGTLKGLEAAHEKWGTRPFQQLITPAFQLAQNGF
SVDRQLALQIENNKEKLAGTAAKEVFLPKGEPIKEGDWLVQKDLAKTFKL
IRSHGSEVFYDGEIGEALAATVQDFGGSMTIEDLQNYGVTEDEPVWGEYK
GYDIASMPPPSSGGLELLQMLKTLDSFDISQYDRRSKEVYHLLAEAMHLS
YADRGAYAGDPEFVEVPMIGLLHPDYIAERSALIDINSVNTNPQPGDPWQ
YEDVDPNYNVIKQNDEKDIGETTHFTVADRWGNLVSYTTTIEQVFGSGIM
VPGYGFMLNNELTDFDARPGGANEVQPNKRPLSSMTPTIVFEDGKPIMSV
GSPGGPTIITSVLQVVLNVMDYEMGLEEAIAEPRIYTNTINSYRYEDGIS
AEVLSELNAMGHRFPSNSELIGNVQSILIDYEKDEYVGVADARRDGASVG
YTRPGKRK

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope, or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, LeuNal, Ala/Glu, and Asp/Gly.

A gamma glutamyl transpeptidase may typically be added in an effective amount such as in the range of 0.01-100 mg of enzyme protein per kg of flour, e.g., 0.1-50 mg of enzyme protein per kg of flour, e.g., 0.5-50 mg of enzyme protein per kg of flour, e.g., 1-50 mg of enzyme protein per kg of flour.

### Additional Enzymes

Optionally, one or more additional enzymes, such as amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucoamylase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase may be used together with the enzyme composition according to the invention.

The additional enzyme(s) may be of any origin, including mammalian, plant, and microbial (bacterial, yeast or fungal) origin.

Suitable commercial alpha-amylase compositions include, e.g., BAKEZYME P 300 (available from DSM) and FUNGAMYL 2500 BG, FUNGAMYL 4000 BG, FUNGAMYL 800 L, FUNGAMYL ULTRA BG and FUNGAMYL ULTRA SG (available from Novozymes NS).

The maltogenic alpha-amylase (EC 3.2.1.133) may be from Bacillus. A maltogenic alpha-amylase from B. stearothermophilus strain NCIB 11837 is commercially available from Novozymes NS under the tradename Novamyl®.

The maltogenic alpha-amylase may also be a variant of the maltogenic alpha-amylase from B. stearothermophilus as disclosed in, e.g., WO1999/043794; WO2006/032281; or WO2008/148845, e.g., Novamyl® 3D.

An anti-staling amylase for use in the invention may also be an amylase (glucan 1,4-alpha-maltotetrahydrolase (EC 3.2.1.60)) from Pseudomonas saccharophilia or variants thereof, such as any of the amylases disclosed in WO1999/050399, WO2004/111217 or WO2005/003339.

The glucoamylase for use in the present invention include the A. niger G1 or G2 glucoamylase (Boel et al. (1984), EMBO J. 3 (5), p. 1097-1102), the A. awamori glucoamylase disclosed in WO 84/02921, or the A. oryzae glucoamylase (Agric. Biol. Chem. (1991), 55 (4), p. 941-949). A suitable commercial glucoamylase is GoldCrust® obtainable from Novozymes A/S.

The glucose oxidase may be a fungal glucose oxidase, in particular an Aspergillus niger glucose oxidase (such as GLUZYME®, available from Novozymes A/S).

The xylanase which may be of microbial origin, e.g., derived from a bacterium or fungus, such as a strain of Aspergillus, in particular of A. aculeatus, A. niger, A. awamori, or A. tubigensis, from a strain of Trichoderma, e.g. T. reesei, or from a strain of Humicola, e.g., H. insolens.

Suitable commercially available xylanase preparations for use in the present invention include PANZEA BG, PENTOPAN MONO BG and PENTOPAN 500 BG (available from Novozymes NS), GRINDAMYL POWERBAKE (available from Danisco), and BAKEZYME BXP 5000 and BAKEZYME BXP 5001 (available from DSM).

The protease may be from Bacillus, e.g., B. amyloliquefaciens. A suitable protease may be Nuetrase® available from Novozymes A/S.

The phospholipase may have phospholipase A1, A2, B, C, D or lysophospholipase activity; it may or may not have lipase activity. It may be of animal origin, e.g., from pancreas, snake venom or bee venom, or it may be of microbial origin, e.g., from filamentous fungi, yeast or bacteria, such as Aspergillus or Fusarium, e.g., A. niger, A. oryzae or F. oxysporum. A preferred lipase/phospholipase from Fusarium oxysporum is disclosed in WO 98/26057. Also, the variants described in WO 00/32758 may be used.

Suitable phospholipase compositions are LIPOPAN F and LIPOPAN XTRA (available from Novozymes NS) or PANAMORE GOLDEN and PANAMORE SPRING (available from DSM).

### Enzyme Treatment

The gamma glutamyl transpeptidase according to the invention is added to the dough ingredients, e.g., indirectly to the dough by adding it to the flour used to prepare the dough, or directly to the dough itself.

The gamma glutamyl transpeptidase may be added to flour or dough in any suitable form, such as, e.g., in the form of a liquid, in particular a stabilized liquid, or it may be added to flour or dough as a substantially dry powder or granulate, so accordingly, we also claim a granulate comprising a gamma glutamyl transpeptidase according to the present invention, and a stabilized liquid comprising a gamma glutamyl transpeptidase according to the present invention.

Granulates may be produced, e.g., as disclosed in U.S. Pat. Nos. 4,106,991 and 4,661,452. Liquid enzyme preparations may, for instance, be stabilized by adding a sugar or sugar alcohol or lactic acid according to established procedures. Other enzyme stabilizers are well-known in the art.

### Pre-Mixes

It will often be advantageous to provide the enzyme(s) used in the treatment of the present invention in admixture with other ingredients used to improve the properties of dough products. These are commonly known in the art as “pre-mixes,” which usually comprise flour.

Hence, in a further aspect, the present invention relates to a premix for improving the quality of dough used to prepare a flat bread product or flat bread products, which premix comprises gamma glutamyl transpeptidase and one or more dough ingredients, in particular flour such as flour from grains, such as, wheat flour, corn flour, rye flour, barley flour, oat flour, rice flour, or sorghum flour, and any combinations thereof.

The premix may also comprise one or more enzymes selected from the group consisting of amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucoamylase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase.

In another embodiment, the present invention relates to a pre-mix comprising the gamma glutamyl transpeptidase of the present invention and flour, such as, flour from grains, such as, wheat flour, corn flour, rye flour, barley flour, oat flour, rice flour, sorghum flour, and any combinations thereof, and one or more additional enzymes, as previously described.

The pre-mix composition may be in liquid form or dry or substantially dry form.

The invention described and claimed herein is not to be limited in scope by the specific embodiments herein disclosed, since these embodiments are intended as illustrations of several aspects of the invention. Any equivalent embodiments are intended to be within the scope of this invention as well as combinations of one or more of the embodiments.

Various references are cited herein, the disclosures of which are incorporated by reference in their entireties. The present invention is further described by the following examples which should not be construed as limiting the scope of the invention.

### EXAMPLES

### Example 1

### Gamma Glutamyl Transpeptidase (GGT) Expression (SEQ ID NO:1)

The Gamma Glutamyl Transpeptidase (GGT) gene was identified in Bacillus licheniformis (ATCC PTA-7992) encoding the GGT protein, SEQ ID NO:1.

Two oligonucleotide primers were designed (SEQ ID NO:3 and SEQ ID NO:4), which allowed PCR amplification of the entire GGT open reading frame (ORF), with the ribosome binding site (RBS) from a Bacillus clausii alkaline protease gene inserted in front of the GGT signal peptide.

The upstream primer, SEQ ID NO:3, incorporated EcoRI and SacI sites in front of the alkaline protease RBS preceding the GGT start.

The downstream primer, SEQ ID NO:4, incorporated MIuI and BamHI following the GGT stop codon.

SEQ ID NO: 3:
5′-GACTGAATTCGAGCTCTATAAAAATGAGGAGGGAACCGAATGAGACG
GTTAGCTTTCTTAG-3′
SEQ ID NO: 4:
5′-GACTGGATCCACGCGTTACTATTTAGCCGATGTCTTAATGT-3′

Chromosomal DNA from B. licheniformis PL1980 (US 8431382) was used as template in a PCR reaction with primers SEQ ID NO:3 and SEQ ID NO:4 in which the annealing temperature was ramped down from 62° C. C to 52° C. in steps of 1° C., then kept constant at 57° C. for 20 cycles.

A PCR fragment of approximately 1.8 kb was obtained, digested with SacI+MIuI, and cloned into the 3.3 kb SacI-MIuI vector fragment from pSJ6814 (described in EP 1766002 B1).

The ligation mixture was transformed into an E. coli laboratory strain by electroporation, selecting ampicillin resistance, and a transformant with the correct DNA sequence of the PCR amplified segment was kept.

The 2.35 kb EcoRI-MIuI segment containing the cryIIIA_stab-ggt construct was excised from the transformant, and ligated to the 4.75 kb MIuI-EcoRI fragment of pSJ6869 (described in US 20140106457).

The ligation mixture was transformed into B. subtilis laboratory strain selecting erythromycin resistance (2 microgram/ml) at 30° C., and a correct transformant was kept.

The correct transformant was transformed into B. subtilis conjugative donor strain PP289-5 (U.S. Pat. No. 6,066,473), resulting in a strain that was used as donor in conjugations to B. licheniformis host strain PP1897-3 (U.S. Pat. No. 8,431,382).

Tetracycline sensitive trans-conjugants were isolated, and colonies with a very weak or absent amylase phenotype were isolated following plasmid integration at 50° C., with ErmR selection. These integrants were propagated at 30° C., to allow plasmid replication and loss, and an amylase negative, erythromycin sensitive strain was obtained.

This Bacillus licheniformis strain was grown as known in the art, and the GGT (SEQ ID NO:1) was recovered as known in the art.

### Example 2

### Gamma Glutamyl Transpeptidase (SEQ ID NO:1) in Baking

Bread was prepared using a straight dough procedure according to below recipe and process conditions. All chemicals applied were food grade. Fungamyl 2500 BG (2500 FAU/g) is available from Novozymes NS.

Gamma glutamyl transpeptidase (GGT—SEQ ID NO:1) may be made as described in Example 1.

TABLE 1
Dough Recipe

| Ingredient | Amount (on flour basis) | |
|---|---|---|
| Flour | 100% | |
| 80% Kolibri (Meneba, NL) | | |
| 20% Victory Landmel N+ | | |
| (HavneMøllerne, DK) | | |
| Tap water | 61% | |
| Yeast (fresh) | 3.4% | |
| Sucrose | 1.5% | |
| Salt | 1.5% | |
| Ascorbic acid | 24 | ppm |
| Calcium Propionate | 0.3% | |
| Fungamyl 2500 BG (Novozymes A/S) | 10 | FAU/kg |

| | |
|---|---|
| Gamma glutamyl transpeptidase | 0; 10; 20; 30; 40 ppm |
| (SEQ ID No: 1) | |

Procedure:

All ingredients were weighed out. Salt, sucrose, yeast, ascorbic acid, calcium propionate and enzyme were added to the mixing bowl. Tap water was weighed out, and the temperature adjusted with ice (to approx. 9-10° C., in order to reach a dough temperature of 27° C. after mixing) and added to the mixing bowl. 2500 g flour (2000 g Kolibri and 500 g Victory) were added to the mixing bowl, and all ingredients were mixed for 3 min at 63 rpm and 7 min at 90 rpm using Spiral mixer (DIOSNA Dierks & Söhne GmbH, DE). The mixed dough was taken out of the mixing bowl and the temperature was controlled, and dough parameters were determined manually (as described in the section—Manual dough evaluation).

The dough was divided into pieces of 450 g each, rounded by hand, where after they rested for 15 min at room temperature covered by plastic. The rested dough pieces were shaped into bread in a sheeter (MO671 MPB-001, Glimek, SE) and transferred to greased 1400 ml pans (Top 230×115×68 mm). The bread was proofed at 32° C. at 86% humidity for 60 min. The proofed bread was baked for 35 min in a deck oven (Piccolo, Wachtel, DE) at 225° C. with steam. The bread was taken out of the pans and allowed to cool to room temperature. Volume of bread was determined as described under volume determination.

TABLE 2
Manual dough evaluation
The dough properties were evaluated approx. 5 min after mixing. A scale between 0-10
was used and dough properties were evaluated relative to a control without addition of
GGT. The control was given the value 5. Details regarding definition, evaluation and
scale is found in below table.

| Parameter | Definition | Evaluation method | Scale |
|---|---|---|---|
| Stickiness | The degree to which | A 3 cm deep cut was | Less sticky 0-4 |
| | a dough adheres to | made in the middle of | Control 5 |
| | one's hands or other | the dough. Stickiness | More sticky 6-10 |
| | surfaces | was evaluated by | |
| | | touch of the fresh cut | |
| | | by the whole palm of | |
| | | a hand | |
| Softness | The degree to, or | Softness was | Less soft 0-4 |
| | ease with, which a | measured by | Control 5 |
| | dough will compress | squeezing and | More soft 6-10 |
| | or resist compression | feeling the dough by | |
| | | hand | |
| Elasticity | The ability of a dough | A piece of dough | Less elastic 0-4 |
| | to resist stretching as | (approx. 30 g) was | Control 5 |
| | well as to return to its | rolled to a dough | More elastic 6-10 |
| | original size and | string of 10 cm which | |
| | shape when the force | was pulled gently in | |
| | is removed | each end to feel the | |
| | | resistance and | |
| | | elasticity | |
| Extensibility | The degree to which a | A piece of dough | Less extensible 0-4 |
| | dough can be | (approx. 30 g) was | Control 5 |
| | stretched without | gently stretched to | More extensible 6-10 |
| | tearing | form a “window” to | |
| | | feel extensibility | |

Volume Determination:

The specific volume was measured using the Volscan profiler 600 (Stable microsystems, UK) running on the Volscan profiler software. Each bread was mounted in the machine. The weight of each loaf was automatically determined with the build-in balance of the Volscan instrument. The volume of each loaf was calculated from a 3D image created by the instrument when each loaf of bread was rotated with a speed of 1.5 revolutions per second while it was scanned with a laser beam taking 3 mm vertical steps per revolution. Specific volume was calculated for each bread according to the following formula:

Specific volume(ml/g)=volume(ml)/weight(g)

The reported value was the average of 2 bread from the same dough.

TABLE 3
Results

| | | 10 ppm | 20 ppm | 30 ppm | 40 ppm |
|---|---|---|---|---|---|
| | Control | GGT | GGT | GGT | GGT |
| Dough stickiness | 5 | 5 | 5 | 5 | 5 |
| Dough softness | 5 | 5 | 5 | 5 | 6 |
| Dough extensibility | 5 | 6 | 7 | 7 | 7 |
| Dough elasticity | 5 | 5 | 5 | 5 | 5 |
| Bread Specific | 4.40 | 4.35 | 4.46 | 4.47 | 4.42 |
| volume (ml/g) | | | | | |

### CONCLUSION

Surprisingly, addition of GGT resulted in a significantly more extensible dough, while no effect on other dough properties was observed. No effect on bread volume was seen.

### Example 3

### Gamma Glutamyl Transpeptidase (SEQ ID NO:2)

A Gamma Glutamyl Transpeptidase (GGT) gene was identified in a Bacillus horikoshii strain.

The Bacillus horikoshii strain was found in New Zealand with a registration date of 15 May 1982.

The Bacillus horikoshii Gamma Glutamyl Transpeptidase, SEQ ID NO:2, has the following mature protein sequence:

QKPVKGSNEVAVGKDGMVSTSHPLASEIGADILRKGGNAMDAAIAVQFAL
NVVEPMMSGIGGGGFMMVYDAETDETTIVNSRERAPAGATPDMFLNPDGS
LIPFQERVRHGNSVGVPGTLKGLEAAHEKWGTRPFQQLITPAFQLAQNGF
SVDRQLALQIENNKEKLAGTAAKEVFLPKGEPIKEGDWLVQKDLAKTFKL
IRSHGSEVFYDGEIGEALAATVQDFGGSMTIEDLQNYGVTEDEPVWGEYK
GYDIASMPPPSSGGLFLLQMLKTLDSFDISQYDRRSKEVYHLLAEAMHLS
YADRGAYAGDPEFVEVPMIGLLHPDYIAERSALIDINSVNTNPQPGDPWQ
YEDVDPNYNVIKQNDEKDIGETTHFTVADRWGNLVSYTTTIEQVFGSGIM
VPGYGFMLNNELTDFDARPGGANEVQPNKRPLSSMTPTIVFEDGKPIMSV
GSPGGPTIITSVLQVVLNVMDYEMGLEEAIAEPRIYTNTINSYRYEDGIS
AEVLSELNAMGHRFPSNSELIGNVQSILIDYEKDEYVGVADARRDGASVG
YTRPGKRK

SEQ ID NO:2 was expressed as an extracellular protein in a Bacillus subtilis host strain as known in the art.

SEQ ID NO:2 showed 68% sequence identity to SEQ ID NO:1.

### Example 4

### Gamma Glutamyl Transpeptidases (SEQ ID NO:1 and SEQ ID NO:2) in Baking

Doughs were prepared using a straight dough procedure according to below recipe and process conditions. All chemicals applied were food grade. Gamma glutamyl transpeptidases (GGT) were added in concentrations as stated in Table 4.

TABLE 4
Dough Recipe:

| Ingredient | Amount (on flour basis) | |
|---|---|---|
| Flour | 100% | |
| 80% Kolibri (Meneba, NL) | | |
| 20% Victory Landmel N+ | | |
| (HavneMøllerne, DK) | | |
| Tap water | 58% | |
| Yeast (fresh) | 4% | |
| Sucrose | 1.5% | |
| Salt | 1.5% | |
| Ascorbic acid | 30 | ppm |

| | |
|---|---|
| Gamma glutamyl transpeptidase | 0; 0.50; 2 mg enzyme |
| (SEQ ID No: 1; SEQ ID No: 2) | protein/kg flour |

Procedure:

All ingredients were weighed out. A stock solution comprising salt, sucrose and ascorbic acid was prepared in tap water and stored on use ice until use. Further a stock solution of yeast was prepared in tap water. Tap water was weighed out; the temperature adjusted (in order to reach a dough temperature of 26° C. after mixing) and then added to the mixing bowl.

100 g flour (80 g Kolibri and 20 g Victory), salt/sugar/ascorbic acid stock solution, GGT and yeast solution were added to the mixing bowl and mixed for 5 min using a 100 g mixer (National MFG Co, Nebraska, Model 100-200A). The mixed dough was taken out of the mixing bowl, rounded to form a ball shape, and the temperature was recorded. Dough parameters were determined manually by hand.

Manual Dough Evaluation:

The dough properties were evaluated approximately 2 min after mixing.

A scale between 0-10 was used, and dough properties were evaluated relative to a control without addition of GGT. The control was run in triplicates and given the value 5.

Softness and elasticity were evaluated first, then the dough ball was cut in half using a sharp knife. Stickiness was measured in the fresh cut. Extensibility was evaluated twice on each dough balls (2 half pieces). Further details regarding definition, evaluation, and scale are found in below Table 5.

TABLE 5
Dough evaluation parameters

| Parameter | Definition | Evaluation method | Scale |
|---|---|---|---|
| Softness | The degree to, or | Softness was | Less soft 0-4 |
| | ease with, which a | measured by | Control 5 |
| | dough will compress | squeezing and | More soft 6-10 |
| | or resist compression | feeling the dough by | |
| | | hand | |
| Elasticity | The ability of a dough | A small piece of | Less elastic 0-4 |
| | to resist stretching as | dough was gently | Control 5 |
| | well as to return to its | pulled from the | More elastic 6-10 |
| | original size and | dough ball using two | |
| | shape when the force | fingers to feel the | |
| | is removed | resistance and | |
| | | elasticity. This was | |
| | | done twice on each | |
| | | dough. | |
| Stickiness | The degree to which | The dough ball was | Less sticky 0-4 |
| | a dough adheres to | cut in half. Stickiness | Control 5 |
| | one's hands or other | was evaluated by | More sticky 6-10 |
| | surfaces | touch of the fresh cut | |
| | | by the whole palm of | |
| | | a hand | |
| Extensibility | The degree to which | A piece of dough | Less extensible 0-4 |
| | a dough can be | (approx. 80 g) was | Control 5 |
| | stretched without | gently stretched to | More extensible 6-10 |
| | tearing | form a “window” to | |
| | | feel extensibility | |

TABLE 6
Results

| | | SEQ ID | SEQ ID | SEQ ID | SEQ ID |
|---|---|---|---|---|---|
| | | NO: 1 | NO: 1 | NO: 2 | NO: 2 |
| | Control | 0.5 mg EP | 2 mg EP | 0.5 mg EP | 2 mg EP |
| Dough | 5 | 5 | 5 | 4 | 4 |
| stickiness | | | | | |
| Dough | 5 | 5 | 5 | 5 | 5 |
| softness | | | | | |
| Dough | 5 | 6 | 6 | 6 | 7 |
| extensibility | | | | | |
| Dough | 5 | 5 | 5 | 5 | 5 |
| elasticity | | | | | |

### CONCLUSION

Both SEQ ID No. 1 and SEQ ID No.2 showed a clear increase on dough extensibility while none or little effect on other dough properties were observed.

1. A method for improving the extensibility of a dough comprising
a) adding a gamma glutamyl transpeptidase (E.C.2.3.2.2) to flour or to a dough comprising a flour; and
b) making the dough.

2. The method according to claim 1, wherein a flattened dough is produced from the dough.

3. The method according to claim 1, wherein the dough is made into an edible product selected from the group consisting of bread, flat bread, crackers, pasta, noodles, laminated baking products, biscuits, baguettes, hamburgers, and pizzas.

4. The method according to claim 1, wherein the flour is selected from the group consisting of wheat flour, corn flour, rye flour, barley flour, oat flour, rice flour, sorghum flour, and a combination thereof.

5. The method according to claim 1, wherein the gamma glutamyl transpeptidase is a bacterial gamma glutamyl transpeptidase.

6. The method according to claim 1, wherein the gamma glutamyl transpeptidase has at least 60% identity with SEQ ID NO:1.

7. The method according to claim 1, wherein the gamma glutamyl transpeptidase is added in an amount of in the range of 0.01-100 mg of enzyme protein per kg of flour.

8. The method according to claim 1, wherein additionally glutathione is added.

9. The method according to claim 1, wherein the dough has an extensibility which is better than the extensibility of a dough which is prepared under the same conditions, but without treatment with a gamma glutamyl transpeptidase.

10. The method according to claim 1, wherein the dough further comprises one or more enzymes selected from the group consisting of amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucoamylase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase.

11. The method according to claim 1, wherein the flat bread are selected from the group consisting of tortillas, pita, Arabic bread, Indian flat bread, wheat and gluten free flat bread.

12. A premix comprising gamma glutamyl transpeptidase (E.C.2.3.2.2) and flour.

13. The premix according to claim 12, wherein the premix further comprises one or more enzymes selected from the group consisting of amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucoamylase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase.

14. (canceled)

15. A composition comprising a gamma glutamyl transpeptidase (E.C.2.3.2.2), wherein the gamma glutamyl transpeptidase has at least 60% identity with SEQ ID NO:1.
